(19) United States
(12) Patent Application Publication
(10) Pub. No.: US 2024/0374138 A1
(43) Pub. Date: Nov. 14, 2024

(54) Computer Implemented Method and System for Classification of Similar Biosignal Curves Detected by an Implantable Medical Device

(71) Applicant: BIOTRONIK SE & Co. KG, Berlin (DE)

(72) Inventors: Thomas DOERR, Berlin (DE); Dirk MUESSIG, West Linn, OR (US)

(73) Assignee: BIOTRONIK SE & Co. KG, Berlin (DE)

(21) Appl. No.: 18/691,295

(22) PCT Filed: Sep. 13, 2022
(86) PCT No.: PCT/EP2022/075354

§ 371 Date: Mar. 12, 2024

Related U.S. Application Data
(60) Provisional application No. 63/246,396, filed on Sep. 21, 2021.

(30) Foreign Application Priority Data
Sep. 29, 2021 (EP) 21199705.1

(51) Int. Cl. A61B 5/00; A61B 5/0538; A61B 5/08; A61B 5/091; A61B 5/29; A61B 5/353; A61B 5/355; A61B 5/361; A61B 5/366

(52) U.S. Cl. CPC A61B 5/0022; A61B 5/0006; A61B 5/0008; A61B 5/0538; A61B 5/0816; A61B 5/091; A61B 5/29; A61B 5/353; A61B 5/355; A61B 5/361; A61B 5/366; A61B 5/686

(57) ABSTRACT

A computer implemented method for classification of similar biosignal curves detected by an implantable medical device, comprising the steps of providing a plurality of biosignal curves by the implantable medical device, storing the detected biosignal curves in a storage medium, applying an algorithm to the plurality of biosignal curves to determine a similarity of each of the plurality of biosignal curves with the plurality of biosignal curves with respect to at least one signal feature according to predetermined similarity criteria, and classifying biosignal curves matching the predetermined similarity criteria. Furthermore, a system for classification of similar biosignal curves detected by an implantable medical device is also provided.

### CROSS-REFERENCE TO RELATED APPLICATIONS

This application is the United States National Phase under 35 U.S.C. § 371 of PCT International Patent Application No. PCT/EP2022/075354, filed on Sep. 13, 2022, which claims the benefit of European Patent Application No. 21199705.1, filed on Sep. 29, 2021, and U.S. Provisional Patent Application No. 63/246,396, filed on Sep. 21, 2021, the disclosures of which are hereby incorporated by reference herein in their entireties.

### TECHNICAL FIELD

The present invention relates to a computer implemented method for classification of similar biosignal curves detected by an implantable medical device.

Furthermore, the present invention relates to a system for classification of similar biosignal curves detected by an implantable medical device.

### BACKGROUND

At present, various methods for automatic reporting and analysis of ECG transmissions are known. These are primarily aimed at automated pre-finding and possible correction of ECG recordings. In cases where patients have a large number of similar arrhythmias, such automated reporting is only partially helpful, since the number of ECG recordings is not significantly reduced in this case.

This can result in a physician or medical provider being burdened with too much data. For example, several hundred ECGs per day can be transmitted by the monitoring system or implant to the physician or medical provider. This causes a high evaluation effort and does not provide any significant clinical added value.

U.S. Publication No. 2007/0112276 A11 discloses a method for differentiating arrhythmic events having different origins, wherein the arrhythmic events are detected in a patient by an implantable medical device, the method comprising receiving electrograms that represent arrhythmic events detected by the implantable medical device, sorting the arrhythmic events into groups based on at least one of similarities between the electrograms or differences between the electrograms, identifying an exemplary arrhythmic event from each group to represent the group from which the exemplary arrhythmic event is identified and distinguishing each exemplary arrhythmic event via an interface display.

The present disclosure is directed toward overcoming one or more of the above-mentioned problems, though not necessarily limited to embodiments that do.

### SUMMARY

It is therefore an object of the present invention to provide an improved method for automatic reporting and analysis of biosignals capable of significantly reducing the number of biosignal recordings that need to be evaluated.

At least the object is solved by a computer implemented method for classification of similar biosignal curves detected by an implantable medical device having the features of claim 1.

Furthermore, at least the object is solved by a system for classification of similar biosignal curves detected by an implantable medical device having the features of claim 14.

Further developments and advantageous embodiments are defined in the dependent claims.

The present invention provides a computer implemented method for classification of similar biosignal curves detected by an implantable medical device.

The method comprises providing a plurality of biosignal curves by the implantable medical device and storing the detected biosignal curves in a storage medium.

The present invention further provides applying an algorithm to the plurality of biosignal curves to determine a similarity of each of the plurality of biosignal curves with the plurality of biosignal curves with respect to at least one signal feature according to predetermined similarity criteria, and classifying biosignal curves matching the predetermined similarity criteria.

The present invention further provides a system for classification of similar biosignal curves detected by an implantable medical device.

The system comprises an implantable medical device for providing a plurality of biosignal curves and a storage medium for storing the detected biosignal curves.

Moreover, the system comprises means for applying an algorithm to the plurality of biosignal curves to determine a similarity of each of the plurality of biosignal curves with the plurality of biosignal curves with respect to at least one signal feature according to predetermined similarity criteria, and means for classifying biosignal curves matching the predetermined similarity criteria.

An idea of the present invention is to significantly reduce the number of biosignal recordings of electronic implants that need to be evaluated. The advantage of the solution according to the present invention is a considerable reduction in the effort required for the evaluation of biosignal recordings of electronic implants.

The method can further be applied to existing implants that acquire biosignal curves usable for the application of the algorithm of the present invention to determine a similarity of each of the plurality of biosignal curves with the plurality of biosignal curves with respect to at least one signal feature according to predetermined similarity criteria and for the classification of biosignal curves matching the predetermined similarity criteria.

If there is sufficient signal similarity, the physician is not shown all the individual signals, but is shown a representative example and the additional information of how many similar of these episodes have been registered.

For example, a patient may have recurrent atrial fibrillation on a regular basis. The physician can thus see from the representative example which signal curve the atrial fibrillation has in this patient and how often this episode has occurred in a given period of time.

According to an aspect of the present invention, the biosignal curves are at least one of ECG and/or IEGM signal curves, respiratory signal curves, in particular a respiratory rhythm and/or a respiratory depth, pressure waveform signal curves, temperature waveform signal curves, tissue and body inductance signal curves and blood flow signal curves. The algorithm present invention for similarity comparison of the recorded biosignal curves is thus advantageously applicable to a wide range of different data types.

According to a further aspect of the present invention, the algorithm for determining the similarity of the biosignal curves performs at least one of a cross-correlation function and an image processing technique with respect to the at least one signal feature, wherein the at least one signal feature is a morphology of a QRS complex, a morphology of a T-wave and/or a morphology of a P-wave of an ECG-signal curve.

The similarity of the biosignal curves can thus advantageously be determined using a host of different techniques and methods best suited to the particular application and/or data type.

According to a further aspect of the present invention, the algorithm, in particular a machine learning algorithm, for determining the similarity of the biosignal curves is configured to classify the biosignal curves matching the predetermined similarity criteria.

The machine learning algorithm is thus advantageously capable of both determining the similarity of the respective biosignal curves and of classifying the biosignal curves matching the predetermined similarity criteria. These two aspects are performed in a single step of generating output data, that is the respective classification result of the bio signal curves to be classified.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

According to a further aspect of the present invention, the similarity of the biosignal curves is determined by a first algorithm, and wherein a classification of biosignal curves matching the predetermined similarity criteria is performed by a second algorithm. This alternative embodiment provides the advantage that a dedicated algorithm most suitable to each task can be applied for similarity comparison and classification respectively.

Moreover, the predetermined similarity criteria are met if a numeric value of the at least one signal feature of a first biosignal curve and at least a second biosignal curve of the plurality of biosignal curves is within a predetermined numeric range. Said numeric range can refer to a degree of match that can, e.g., be expressed in terms of a percentage. The degree of match may indicate a type of episode, a length of a particular episode, a rhythm of occurrence of each episode, a signal morphology, etc.

According to a further aspect of the present invention, a classification result of biosignal curves matching the predetermined similarity criteria is sent to a communication device of a healthcare provider, wherein the classification result comprises at least one designated class, a number of similar episodes of the classified biosignal curves and a time interval, in which the classified number of similar episodes occurred.

Arranging the classified results in this manner advantageously provides an efficient overview for the physician and/or healthcare provider in charge of diagnosing the patient.

According to a further aspect of the present invention, the application of the algorithm to the plurality of biosignal curves and/or the classification of the biosignal curves matching the predetermined similarity criteria is performed by an executable program embedded within the implantable medical device and/or operating on a remote server, and wherein the biosignal curves are transmitted to the remote server via a patient communication device or smartphone.

Operating the algorithm on the implantable medical device thereby advantageously provides an energetic benefit for the battery life of the implantable medical device since the amount of data transmitted to the remote server is reduced considerably.

According to a further aspect of the present invention, the biosignal curves are acquired by the implantable medical device at predetermined intervals and/or on request, in particular as a wide-field ECG between electrodes and a housing of the implantable medical device. Such predetermined intervals can, e.g., be daily or weekly depending on a patient condition and the specific need for a frequency of monitoring said condition. In any case, said predetermined intervals pose a considerable advantage over less frequent biosignal data acquisition at aftercare visits to the medical provider thus enabling a more timely and accurate diagnosis of a patient condition.

According to a further aspect of the present invention, the implantable medical device is a diagnostic implant, in particular a cardiac rhythm monitor, a pressure sensor implant or a vital sign acquisition implant for acquiring respiratory data and/or temperature data, a therapeutic implant, in particular a cardiac pacemaker or a defibrillator, or a neurostimulator. The method according to the present invention can thus advantageously be applied to a wide variety of different implantable medical devices.

According to a further aspect of the present invention, at least one classification result of biosignal curves matching the predetermined similarity criteria is sent to the patient communication device or smartphone, in particular by the implantable medical device or the remote server, wherein the at least one classification result is accessible to a user of the implantable medical device via the patient communication device or smartphone.

The user of the implantable medical device can thus be informed about a frequency and type of medical episode. This could, e.g., prompt the user to schedule an appointment with the healthcare provider sooner or later than originally planned depending on the data communicated to the user.

According to a further aspect of the present invention, based on the classification of the biosignal curves matching the predetermined similarity criteria, operating parameters of the implantable medical device are adjusted. The medical data of the patient analyzed and classified in this fashion can thus be advantageously used to change specific parameters of the implantable medical device.

Such parameters, e.g., could be to change the frequency of data acquisition of a particular biosignal or to enable or disable the acquisition of other bio signals in order to enhance the accuracy of diagnosis of a certain condition and/or complement the existing medical data of the patient with additional data points.

According to a further aspect of the present invention, the storage medium for storing the detected biosignal curves is arranged in the implantable medical device and/or in a remote server, wherein the biosignal curves are transmittable to the remote server via a patient communication device or smartphone.

The acquired biosignal curves can thus advantageously be stored in the storage medium arranged in either the implantable medical device or in the remote server. Which of these strategies is given preference may be determined based on factors such as remaining battery life of the implantable medical device and/or frequency of transmission of the biosignal curves.

The herein described features of the system for classification of similar biosignal curves detected by an implantable medical device are also disclosed for the computer-implemented method for classification of similar biosignal curves detected by an implantable medical device and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a diagram of a system for classification of similar biosignal curves detected by an implantable medical device according to a preferred embodiment of the present invention; and

FIG. 2 shows a flowchart of a computer implemented method for classification of similar biosignal curves detected by an implantable medical device according to a preferred embodiment of the present invention.

### DETAILED DESCRIPTION

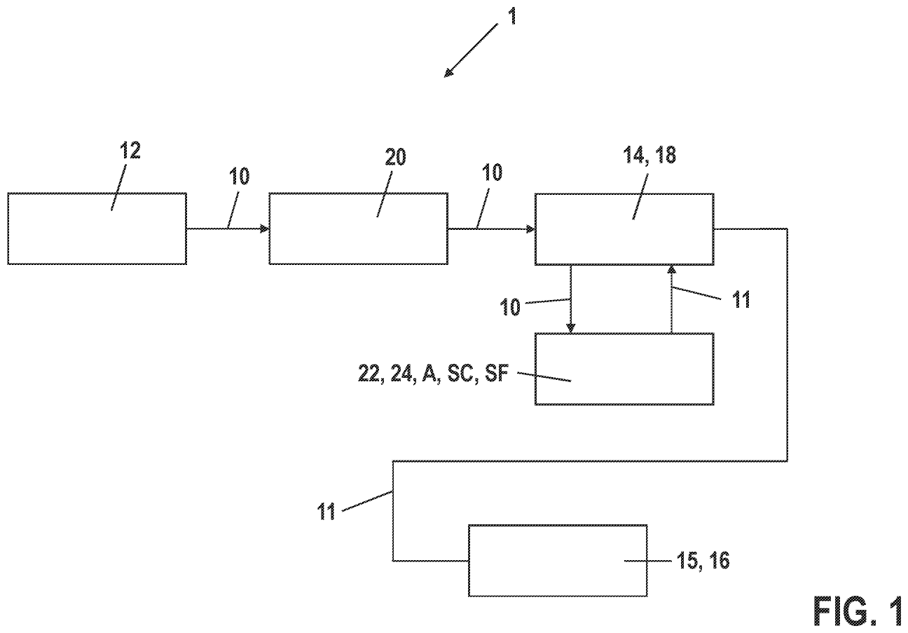

The system 1 shown in FIG. 1 comprises an implantable medical device 12 for providing a plurality of biosignal curves 10 and a storage medium 14 for storing the detected biosignal curves 10.

Moreover, the system 1 comprises means 22 for applying an algorithm A to the plurality of biosignal curves 10 to determine a similarity of each of the plurality of biosignal curves 10 with the plurality of biosignal curves 10 with respect to at least one signal feature SF according to predetermined similarity criteria SC, and means 24 for classifying biosignal curves 10 matching the predetermined similarity criteria SC.

In addition, the storage medium 14 for storing the detected biosignal curves 10 is arranged in a remote server, wherein the biosignal curves 10 are transmittable to the remote server 18 via a patient communication device 20 or smartphone. Alternatively, or additionally, the storage medium 14 can be arranged in the implantable medical device 12.

The biosignal curves 10 are at least one of ECG and/or IEGM signal curves, respiratory signal curves, in particular a respiratory rhythm and/or a respiratory depth, pressure waveform signal curves, temperature waveform signal curves, tissue and body inductance signal curves and blood flow signal curves.

Furthermore, the algorithm A for determining the similarity of the biosignal curves 10 performs at least one of a cross-correlation function and an image processing technique with respect to the at least one signal feature SF. Moreover, the at least one signal feature SF is a morphology of a QRS complex, a morphology of a T-wave and/or a morphology of a P-wave of an ECG-signal curve. The algorithm A is run either on the remote server 18 and/or a further computing device connected to the remote server 18 via a network connection.

The algorithm A, in particular a machine learning algorithm A, for determining the similarity of the biosignal curves 10 is configured to classify the biosignal curves 10 matching the predetermined similarity criteria SC.

Said machine learning algorithm A is trained by receiving a first training data set comprising pre-acquired biosignal curves 10 captured by an implantable medical device 12, and receiving a second training data set representing a classification of biosignal curves 10 matching predetermined similarity criteria SC.

In addition, the training method comprises training the machine learning algorithm A by an optimization algorithm A which calculates an extreme value of a loss function for classification of the biosignal curves 10 matching the predetermined similarity criteria SC from the biosignal curves 10 pre-acquired by the implantable medical device 12.

Alternatively, the similarity of the biosignal curves 10 is determined by a first algorithm and a classification of biosignal curves 10 matching the predetermined similarity criteria SC is performed by a second algorithm.

The predetermined similarity criteria SC are met if a numeric value of the at least one signal feature SF of a first biosignal curve and at least a second biosignal curve of the plurality of biosignal curves 10 is within a predetermined numeric range.

A classification result 11 of biosignal curves 10 matching the predetermined similarity criteria SC is sent to a communication device 16 of a healthcare provider. The classification result 11 comprises at least one designated class, a number of similar episodes of the classified biosignal curves 10 and a time interval, in which the classified number of similar episodes occurred. Said classification result 11 is accessible to the physician or healthcare provider via a front-end application 15.

The application of the algorithm A to the plurality of biosignal curves 10 and/or the classification of the biosignal curves 10 matching the predetermined similarity criteria SC is performed by an executable program embedded within the implantable medical device 12 and/or operating on a remote server 18. Furthermore, the biosignal curves 10 are transmitted to the remote server 18 via a patient communication device 20 or smartphone.

The biosignal curves 10 are acquired by the implantable medical device 12 at predetermined intervals and/or on request, in particular as a wide-field ECG between electrodes and a housing of the implantable medical device 12.

The implantable medical device 12 is preferably a diagnostic implant, in particular a cardiac rhythm monitor. Alternatively, the implantable medical device 12 can be a pressure sensor implant or a vital sign acquisition implant for acquiring respiratory data and/or temperature data, a therapeutic implant, in particular a cardiac pacemaker or a defibrillator, or a neurostimulator.

At least one classification result 11 of biosignal curves 10 matching the predetermined similarity criteria SC is sent to the communication device 16 or smartphone, in particular by the implantable medical device 12 or the remote server 18. Moreover, the at least one classification result 11 is accessible to a user of the implantable medical device 12 via the communication device 16 or smartphone.

Based on the classification of the biosignal curves 10 matching the predetermined similarity criteria SC, operating parameters of the implantable medical device are adjusted.

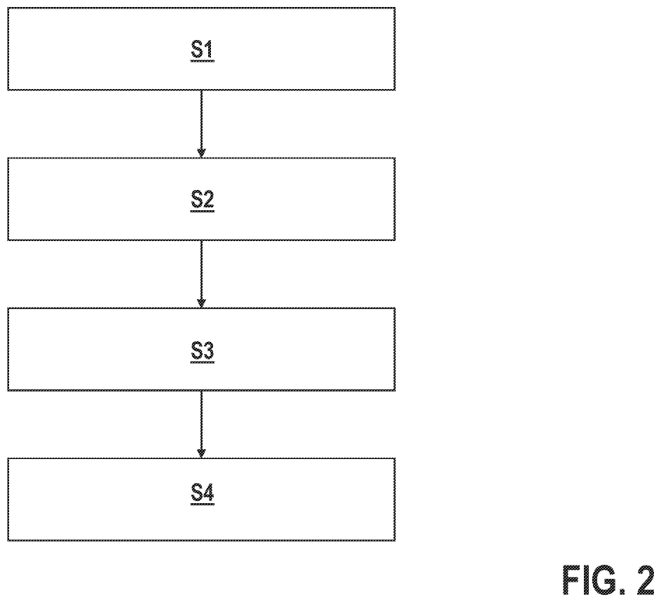

FIG. 2 shows a flowchart of a computer implemented method for classification of similar biosignal curves detected by an implantable medical device according to a preferred embodiment of the present invention.

The method shown in FIG. 2 comprises providing a plurality of biosignal curves 10 by the implantable medical device 12 (step S1) and storing the detected biosignal curves 10 in a storage medium 14 (step S2).

Furthermore, the method comprises applying an algorithm A to the plurality of biosignal curves 10 to determine a similarity of each of the plurality of biosignal curves 10 with the plurality of biosignal curves 10 with respect to at least one signal feature SF according to predetermined similarity criteria SC (step S3), and classifying biosignal curves 10 matching the predetermined similarity criteria SC (step S4).

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
10 biosignal curves
11 classification result
12 implantable medical device
14 storage medium
15 front-end application
16 communication device
18 remote server
20 patient communication device
22, 24 means
A algorithm
SC similarity criteria
SF signal feature
S1-S4 method steps

1. Computer-implemented method for classification of similar biosignal curves detected by an implantable medical device, comprising the following steps:
providing a plurality of biosignal curves by the implantable medical device;
storing the detected biosignal curves in a storage medium;
applying an algorithm to the plurality of biosignal curves to determine a similarity of each of the plurality of biosignal curves with the plurality of biosignal curves with respect to at least one signal feature according to predetermined similarity criteria; and
classifying biosignal curves matching the predetermined similarity criteria.

2. Computer-implemented method of claim 1, wherein the biosignal curves are at least one of ECG and/or IEGM signal curves, respiratory signal curves, in particular a respiratory rhythm and/or a respiratory depth, pressure waveform signal curves, temperature waveform signal curves, tissue and body inductance signal curves and blood flow signal curves.

3. Computer-implemented method of claim 1, wherein the algorithm for determining the similarity of the biosignal curves performs at least one of a cross-correlation function and an image processing technique with respect to the at least one signal feature, wherein the at least one signal feature is a morphology of a QRS complex, a morphology of a T-wave and/or a morphology of a P-wave of an ECG-signal curve.

4. Computer-implemented method of claim 1, wherein the algorithm, in particular a machine learning algorithm, for determining the similarity of the biosignal curves is configured to classify the biosignal curves matching the predetermined similarity criteria.

5. Computer-implemented method of claim 1, wherein the similarity of the biosignal curves is determined by a first algorithm, and wherein a classification of biosignal curves matching the predetermined similarity criteria is performed by a second algorithm.

6. Computer-implemented method of claim 1, wherein the predetermined similarity criteria are met if a numeric value of the at least one signal feature of a first biosignal curve and at least a second biosignal curve of the plurality of biosignal curves is within a predetermined numeric range.

7. Computer-implemented method of claim 1, wherein a classification result of biosignal curves matching the predetermined similarity criteria is sent to a communication device of a healthcare provider, wherein the classification result comprises at least one designated class, a number of similar episodes of the classified biosignal curves and a time interval, in which the classified number of similar episodes occurred.

8. Computer-implemented method of claim 1, wherein the application of the algorithm to the plurality of biosignal curves and/or the classification of the biosignal curves matching the predetermined similarity criteria is performed by an executable program embedded within the implantable medical device and/or operating on a remote server, and wherein the biosignal curves are transmitted to the remote server via a patient communication device or smartphone.

9. Computer-implemented method of claim 1, wherein the biosignal curves are acquired by the implantable medical device at predetermined intervals and/or on request, in particular as a wide-field ECG between electrodes and a housing of the implantable medical device.

10. Computer-implemented method of claim 1, wherein the implantable medical device is a diagnostic implant, in particular a cardiac rhythm monitor, a pressure sensor implant or a vital sign acquisition implant for acquiring respiratory data and/or temperature data, a therapeutic implant, in particular a cardiac pacemaker or a defibrillator, or a neurostimulator.

11. Computer-implemented method of claim 8, wherein at least one classification result of biosignal curves matching the predetermined similarity criteria is sent to the patient communication device or smartphone, in particular by the implantable medical device or the remote server, wherein the at least one classification result is accessible to a user of the implantable medical device via the patient communication device or smartphone.

12. Computer-implemented method of claim 1, wherein based on the classification of the biosignal curves matching the predetermined similarity criteria, operating parameters of the implantable medical device are adjusted.

13. System for classification of similar biosignal curves detected by an implantable medical device, comprising:
an implantable medical device for providing a plurality of biosignal curves;
a storage medium for storing the detected biosignal curves;
means for applying an algorithm to the plurality of biosignal curves to determine a similarity of each of the plurality of biosignal curves with the plurality of biosignal curves with respect to at least one signal feature according to predetermined similarity criteria; and
means for classifying biosignal curves matching the predetermined similarity criteria.

14. System of claim 13, wherein the storage medium for storing the detected biosignal curves is arranged in the implantable medical device and/or in a remote server, wherein the biosignal curves are transmittable to the remote server via a patient communication device or smartphone.

15. Computer program with program code to perform the method of claim 1 when the computer program is executed on a computer.
